(19) United States
(12) Patent Application Publication
(10) Pub. No.: US 2024/0002328 A1
(43) Pub. Date: Jan. 4, 2024

(54) SELENIUM-BASED CATALYST SYSTEM FOR PREPARING CARBONATE DERIVATIVE, AND METHOD FOR PREPARING CARBONATE DERIVATIVE BY USING SAME

(71) Applicant: KOREA INSTITUTE OF INDUSTRIAL TECHNOLOGY, Cheonan-si (KR)

(72) Inventors: Yong Jin KIM, Yongin-si (KR); Jayeon BAEK, Cheonan-si (KR); Hye Jin LEE, Cheonan-si (KR)

(21) Appl. No.: 18/255,829

(22) PCT Filed: Jul. 8, 2021
(86) PCT No.: PCT/KR2021/008715

§ 371 Date: Jun. 2, 2023

(30) Foreign Application Priority Data
Dec. 4, 2020 (KR) 10-2020-0168049

(51) Int. Cl. C07C 67/40; B01J 31/02; B01J 27/057

(52) U.S. Cl. CPC C07C 67/40; B01J 31/0244; B01J 27/0573

(57) ABSTRACT

The present invention relates to a catalyst system for preparing a carbonate derivative, comprising selenium (Se) and a pyridine amine compound represented by structural formula 1. The catalyst system for preparing a carbonate derivative, and a method for preparing a carbonate derivative by using same, of the present invention, allow an alcohol to undergo oxidative carbonylation by using a selenium-based catalyst system, and thus are more economical than a conventional carbonylation process and can obtain a dialkyl carbonate in feasible yields.

### TECHNICAL FIELD

The present disclosure relates to a catalyst system for preparing a carbonate derivative and a method of preparing a carbonate derivative using the same system and, more particularly, to a selenium-based catalyst system for preparing a carbonate derivative and a method of preparing a carbonate derivative using the same system.

### BACKGROUND ART

Dialkyl carbonates (DACs) are attracting attention because they are environmentally benign and can have a wide range of applications, such as aprotic solvents, monomers for polycarbonates, and alkylating agents. In addition, these dialkyl carbonates are used as dialkyl carbonate electrolyte solvents and gasoline additives, and can be easily prepared by the reaction of phosgene with alcohol.

However, in the phosgenation, problems caused due to the use of highly toxic phosgene occur. To replace such phosgenation, several processes have been developed, including transesterification, methyl nitrile carbonylation, alcoholysis of urea, oxidative carbonylation, and carboxylation. Among these processes, the synthesis of DAC by carboxylation with CO₂ is the most preferable from environmental and economic viewpoints. However, economically feasible processes using CO₂ as a raw material are yet to be developed. In addition, the reaction has problems, such as by-product generation during the reaction and complicated processes.

On the other hand, a method of producing dimethyl carbonate through oxidative carbonylation using a copper-based catalyst, such as CuCl, has been commercialized by Enichem (see U.S. Pat. No. 5,536,864). Since then, many efforts have been made to improve the activities of copper-based catalysts. A successful example is the case where PdCl₂ and ammonium salts are used together with CuCl₂. Dow Chemical has also issued several patents for the use of activated carbon-supported copper catalysts (see U.S. Pat. Nos. 5,004,827, 4,625,044).

Nevertheless, the catalyst systems reported so far regarding oxidative carbonylation still need improvement in terms of activity, selectivity, catalyst recovery, and corrosion.

### DISCLOSURE

### Technical Problem

An objective of the present disclosure is to provide a catalyst system capable of obtaining a carbonate derivative, such as dialkyl carbonates or dialkoxyalkyl carbonates, economically and feasibly at a high yield, compared to existing carbonylation processes, by performing an oxidative carbonylation process of alcohol using a selenium-based catalyst system, and a method of preparing a carbonate derivative using the same system.

Another objective of the present disclosure is to provide a catalyst system for preparing a carbonate derivative by using a selenium-based catalyst system capable of maintaining activity even when reused multiple times, and a method of preparing a carbonate derivative using the same system.

### Technical Solution

According to one aspect of the present disclosure, provided is a catalyst system for preparing a carbonate derivative, the catalyst system containing selenium (Se); and a pyridine amine compound represented by Structural Formula 1.

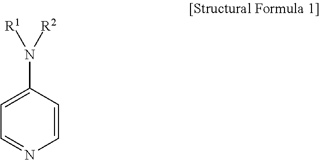

In Structural Formula 1,

R¹ is an alkyl group having 1 to 3 carbon atoms, and

R² is an alkyl group having 1 to 3 carbon atoms.

The pyridine amine compound may be 4-dimethylaminopyridine (DMAP).

In the catalyst system, a molar ratio (DMAP/Se) of 4-dimethylaminopyridine (DMAP) to selenium (Se) may be in a range of 0.5 to 5.

The catalyst system may further contain a promoter.

The promoter may include at least one selected from the group consisting of diphenyl diselenide (Ph₂Se₂), dimethyl diselenide (Me₂Se₂), dibenzyl diselenide (Bz₂Se₂), and diphenyl selenide (Ph₂Se).

The promoter may include diphenyl diselenide (Ph₂Se₂).

In the catalyst system, a molar ratio (promoter/Se) of the promoter to selenium (Se) may be in a range of 0.5 to 1.5.

The carbonate derivative may be a compound represented by Structural Formula 2.

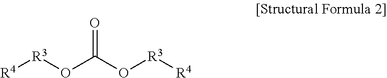

In Structural Formula 2,

R³ is each independently an alkylene group having 1 to 3 carbon atoms,

R⁴ is each independently a hydrogen atom or

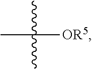

and

R⁵ is each independently an alkyl group having 1 to 3 carbon atoms.

The carbonate derivative may be bis(2-methoxyethyl) carbonate (BMEC).

According to another aspect of the present disclosure, provided is a method of preparing a carbonate derivative, the method including causing reactants including Compound 3, carbon monoxide, and oxygen to react according to Reaction Formula 1 using a catalyst system to prepare Compound 2.

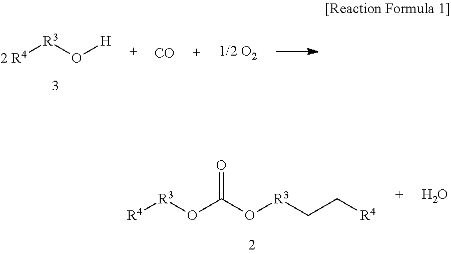

In Reaction Formula 1,

R³ is each independently an alkylene group having 1 to 3 carbon atoms,

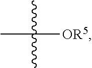

R⁴ is each independently a hydrogen atom or, and

R⁵ is each independently an alkyl group having 1 to 3 carbon atoms.

The catalyst system may contain selenium (Se) and a pyridine amine compound represented by the Structural Formula 1.

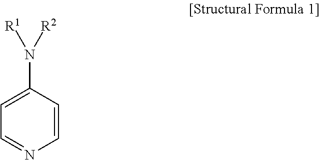

In Structural Formula 1,

R¹ is an alkyl group having 1 to 3 carbon atoms, and

R² is an alkyl group having 1 to 3 carbon atoms.

The pyridine amine compound may be 4-dimethylaminopyridine (DMAP).

The catalyst system may further contain a promoter.

The promoter may include at least one selected from the group consisting of diphenyl diselenide (Ph₂Se₂), dimethyl diselenide (Me₂Se₂), dibenzyl diselenide (Bz₂Se₂), and diphenyl selenide (Ph₂Se).

The promoter may include diphenyl diselenide (Ph₂Se₂).

The reaction may be performed at a temperature in a range of 40° C. to 150° C.

The reaction may be performed at a pressure in a range of 1 to 10 MPa.

The reaction may be performed for a duration in a range of 30 minutes to 5 hours.

### Advantageous Effects

A catalyst system for preparing a carbonate derivative and a method of preparing a carbonate derivative using the same system of the present disclosure enable a dialkyl carbonate to be obtained economically and feasibly at a high yield, compared to existing carbonylation processes, by performing an oxidative carbonylation process of alcohol using a selenium-based catalyst system.

In addition, the catalyst system for preparing the carbonate derivative and the method of preparing the carbonate derivative using the same system of the present disclosure have an effect of maintaining catalytic activity even when reused multiple times by using the selenium-based catalyst system.

Furthermore, while existing CuCl or CuCl₂-based catalyst systems contain halogen elements and thus cause corrosion in a reactor, the selenium-based catalyst system does not have such a problem.

### DESCRIPTION OF DRAWINGS

FIG. 1 is a diagram illustrating structures of base substances of L1 to L12.

### BEST MODE

While including various transformations in terms of reaction conditions and various embodiments, the present disclosure will be described in detail with reference to specific embodiments of the present disclosure. However, this is not intended to limit the embodiments according to the concept of the present disclosure to a specific disclosed form, and should be understood to include all changes, equivalents, or substitutes included in the spirit and technical scope of the present disclosure. In describing the present disclosure, if it is determined that a detailed description of a related known function or configuration may unnecessarily obscure the gist of the present disclosure, the detailed description will be omitted.

In addition, terms used in the specification, “first”, “second”, etc., may be used to describe various components, but the components are not to be construed as being limited to the terms. These terms are used only for the purpose of distinguishing a component from another component. For example, without departing from the scope of the present disclosure, a first component may be referred to as a second component, and a second component may be also referred to as a first component.

In addition, when a component is referred to as being “formed”, “positioned”, or “laminated” on another component, it may be formed, positioned, or laminated directly or attached to the front or one surface on the surface of the other component, but it will be understood that intervening elements may be present therebetween.

The singular expression includes the plural expression unless the context clearly indicates otherwise. It will be further understood that the terms “comprises”, “includes”, or “has” when used in this specification specify the presence of stated features, regions, integers, steps, operations, elements, and/or components, but do not preclude the presence or addition of one or more other features, regions, integers, steps, operations, elements, components, and/or combinations thereof.

Hereinafter, a catalyst system for preparing a carbonate derivative of the present disclosure will be described.

The present disclosure provides a catalyst system for preparing a carbonate derivative, the system containing selenium (Se) and a pyridine amine compound represented by Structural Formula 1.

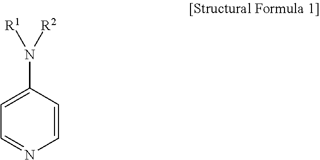

In Structural Formula 1,

R¹ is an alkyl group having 1 to 3 carbon atoms, and

R² is an alkyl group having 1 to 3 carbon atoms.

The pyridine amine compound may be 4-dimethylaminopyridine (DMAP).

In the catalyst system, a molar ratio (DMAP/Se) of 4-dimethylaminopyridine (DMAP) to selenium (Se) may be in a range of 0.5 to 5, preferably, 2 to 3. When the molar ratio (DMAP/Se) of 4-dimethylaminopyridine (DMAP) to selenium (Se) is lower than 0.5, the yield is low, which is undesirable. Even when the molar ratio exceeds 5, the yield is somewhat low, which is undesirable.

The catalyst system may further contain a promoter.

The promoter may include at least one selected from the group consisting of diphenyl diselenide (Ph₂Se₂), dimethyl diselenide (Me₂Se₂), dibenzyl diselenide (Bz₂Se₂), and diphenyl selenide (Ph₂Se), and preferably includes diphenyl diselenide (Ph₂Se₂).

In the catalyst system, a molar ratio (promoter/Se) of the promoter to selenium (Se) may be in a range of 0.5 to 1.5, preferably, 0.8 to 1.2, and more preferably, 1.

The carbonate derivative may be a compound represented by Structural Formula 2.

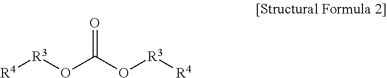

In Structural Formula 2,

R³ is each independently an alkylene group having 1 to 3 carbon atoms,

R⁴ is each independently a hydrogen atom or

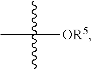

and

R⁵ is each independently an alkyl group having 1 to 3 carbon atoms.

The carbonate derivative may be bis(2-methoxyethyl) carbonate (BMEC).

In addition, the present disclosure provides a method of preparing a carbonate derivative, the method including causing reactants including Compound 3, carbon monoxide, and oxygen to react according to Reaction Formula 1 using a catalyst system to prepare Compound 2.

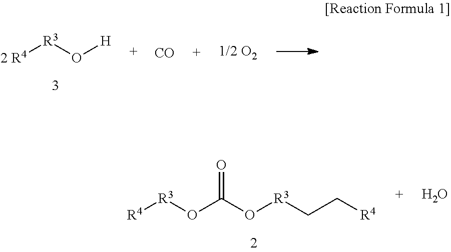

In Reaction Formula 1,

R³ is each independently an alkylene group having 1 to 3 carbon atoms,

R⁴ is each independently a hydrogen atom or

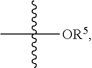

and

R⁵ is each independently an alkyl group having 1 to 3 carbon atoms.

The catalyst system may contain selenium (Se) and a pyridine amine compound represented by the Structural Formula 1.

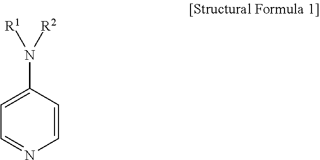

In Structural Formula 1,

R¹ is an alkyl group having 1 to 3 carbon atoms, and

R² is an alkyl group having 1 to 3 carbon atoms.

The pyridine amine compound may be 4-dimethylaminopyridine (DMAP).

The catalyst system may further contain a promoter.

The promoter may include least one selected from the group consisting of diphenyl diselenide (Ph₂Se₂), dimethyl diselenide (Me₂Se₂), dibenzyl diselenide (Bz₂Se₂), and diphenyl selenide (Ph₂Se), and preferably includes diphenyl diselenide (Ph₂Se₂).

In Reaction Formula 1, Compound 3 may include 2-methoxyethanol (MEG).

The reaction may be performed at a temperature in a range of 40° C. to 150° C., preferably, 70° C. to 100° C., and more preferably, 70° C. to 90° C. When performing the reaction at a temperature lower than 40° C., the BMEC yield is low, which is undesirable. When performing the reaction at a temperature exceeding 150° C., the amount of by-products is increased, which is undesirable.

The reaction may be performed at a pressure in a range of 1 to 10 MPa, preferably, 3 to 8 MPa, and more preferably, 5 to 7 MPa. When performing the reaction at a pressure lower than 1 MPa, the BMEC yield is low, which is undesirable. When performing the reaction at a pressure exceeding 10 MPa, the amount of by-products is increased, which is undesirable.

The reaction may be performed for a duration in a range of 30 minutes to 5 hours, preferably, 40 minutes to 3 hours, and more preferably, 50 minutes to 2 hours. When performing the reaction for less than 30 minutes, the BMEC yield is low, which is undesirable. When performing the reaction for more than 4 hours, the amount of by-products is increased, which is undesirable.

In Reaction Formula 1, a volume ratio of carbon monoxide (CO) and oxygen (O₂) (O₂/CO, v/v) may be in a range of 0.01 to 1, preferably, 0.1 to 0.3, and more preferably, 0.2 to 0.3. There may be a risk of explosion when the oxygen partial pressure exceeds 30%.

Dialkyl carbonates may be prepared by an oxidative carbonylation process of alcohol in the presence of the catalyst system for preparing the carbonate derivative according to the present disclosure, and the above reaction may be continuously performed in a reactor.

When performing the oxidative carbonylation of alcohol in the presence of the catalyst system according to the present disclosure, the catalyst system may be converted to elemental selenium and DMAP, thereby preparing bis(2-methoxyethyl) carbonate economically at a feasible yield, compared to existing carbonylation processes.

### MODE FOR INVENTION

### Example

Hereinafter, all chemicals including alcohols and catalysts used in the synthesis of dialkyl carbonates were purchased from Aldrich Chemical, and O₂ with a purity of 99.9% was purchased from Shinyang Gas, Korea. In addition, all catalysts were dried in vacuo before use. The present disclosure will be described in more detail with examples. However, these examples are for illustrative purposes and the scope of the present disclosure is not limited thereby.

[Catalyst System]

Catalyst System 1-1: Se/DMAP (1:3)

Selenium (Se, 0.030 g, 0.375 mmol) and 4-dimethylaminopyridine (DMAP, 0.375 g, 1.125 mmol) were used for Catalyst system 1-1.

Catalyst System 2: Se Only

Selenium (Se) was used for Catalyst system 2.

Catalyst System 3: DMAP Only

4-Dimethylaminopyridine (DMAP) was used for Catalyst system 3.

Catalyst System 4-1: Se/Ph₂Se₂/DMAP (1:1:3)

Selenium (Se, 0.030 g, 0.375 mmol), 4-dimethylaminopyridine (DMAP, 0.375 g, 1.125 mmol), and Ph₂Se₂ (0.117 g, 0.375 mmol) were used for Catalyst system 4-1.

Catalyst System 5: Se/pH₂Se₂ (1:1)

Selenium (Se, 0.030 g, 0.375 mmol) and Ph₂Se₂ (0.117 g, 0.375 mmol) were used for Catalyst system 5.

Catalyst System 6: pH₂Se₂/DMAP (1:3)

4-Dimethylaminopyridine (DMAP, 0.375 g, 1.125 mmol) and Ph₂Se₂ (0.117 g, 0.375 mmol) were used for Catalyst system 6.

The catalyst systems, according to Examples 1-1 to 1-6, 2, 3, 4-1 to 4-6, 5, and 6, were used by varying molar ratios and compositions of the catalyst systems. Compositions of the catalyst systems are shown in Table 1 below.

TABLE 1

| | Active | | |
|---|---|---|---|
| Catalyst system | species | Promoter | Molar ratio |
| Catalyst system 1-1 | Se/DMAP | — | Se/DMAP (1:3) |
| Catalyst system 1-2 | Se/DMAP | — | Se/DMAP (1:1) |
| Catalyst system 1-3 | Se/DMAP | — | Se/DMAP (1:5) |
| Catalyst system 1-4 | Se/DMAP | — | Se/DMAP (1:2) |
| Catalyst system 1-5 | Se/DMAP | — | Se/DMAP (1:4) |
| Catalyst system 1-6 | Se/DMAP | — | Se/DMAP (2:1) |
| Catalyst system 2 | Se | — | — |
| Catalyst system 3 | DMAP | — | — |
| Catalyst system 4-1 | Se/DMAP | Ph₂Se₂ | Se/Ph₂Se₂/DMAP |
| | | | (1:1:3) |
| Catalyst system 4-2 | Se/DMAP | Ph₂Se₂ | Se/Ph₂Se₂/DMAP |
| | | | (1:1:1) |
| Catalyst system 4-3 | Se/DMAP | Ph₂Se₂ | Se/Ph₂Se₂/DMAP |
| | | | (1:1:5) |
| Catalyst system 4-4 | Se/DMAP | Me₂Se₂ | Se/Me₂Se₂/DMAP |
| | | | (1:1:3) |
| Catalyst system 4-5 | Se/DMAP | Bz₂Se₂ | Se/Bz₂Se₂/DMAP |
| | | | (1:1:3) |
| Catalyst system 4-6 | Se/DMAP | Ph₂Se | Se/Ph₂Se/DMAP |
| | | | (1:1:3) |
| Catalyst system 5 | Se | Ph₂Se₂ | Se/Ph₂Se₂ (1:1) |
| Catalyst system 6 | DMAP | Ph₂Se₂ | Ph₂Se₂/DMAP (1:3) |

[Oxidative Carbonylation of 2-Methoxyethanol (MEG)]

### Examples 1 to 16

Oxidative carbonylation was performed in a 100-mL Teflon-lined stainless steel reactor equipped with a magnetic stirrer, a thermocouple, and an electric heater. The reactor was charged with an appropriate alcohol and a catalyst system. The reactor was compressed using a mixed gas of CO/O₂ (v/v=80/20) at room temperature and then heated to a desired temperature with vigorous stirring. The reactor was further compressed at a specified temperature at a desired pressure and then kept remained throughout the reaction using a gas reservoir equipped with a high-pressure regulator and a pressure transducer. After completion of the reaction, the reactor was cooled to room temperature, and bis(2-methoxyethyl) carbonate was obtained by oxidative carbonylation. The conversion rate of alcohol, in addition to the DAC yield and selectivity, was obtained by GC-FID and calculated using Equations 1 to 3.

$$Conversion of alcohol ( \% ) = \frac{Alcohol reacted ( by GC - FID ) ( mmol )}{Alcohol charged ( mmol )} \times 100 \% \qquad [ Equation 1 ]$$

$$Yeild of DAC ( \% ) = \frac{2 \times DAC produced ( by GC - FID ) ( mmol )}{Alcohol charged ( mmol )} \times 100 \% \qquad [ Equation 2 ]$$

$$Selectivity of DAC ( \% ) = \frac{Yeild of DAC ( \% )}{Conversion of alcohol ( \% )} \times 100 \% \qquad [ Equation 3 ]$$

Bis(2-methoxyethyl) carbonate was obtained in the same manner as in the above method by varying the following conditions: a molar ratio of 2-methoxyethanol (MEG) and selenium (Se), a catalyst system used, a reaction temperature (T, ° C.), a reaction pressure (P, MPa), and a reaction time (t, h). Experimental conditions, conversion rate (Conv.), yield, and turnover frequency (TOF) of Examples 1 to 16 are specifically described in Table 2 below.

TABLE 2

| | Molar ratio | Catalyst system | | T | P | t | Conv. | Yield | TOF |
|---|---|---|---|---|---|---|---|---|---|
| Entry | (MEG/Se) | (molar ratio) | | (° C.) | (MPa) | (h) | (%) | (%) | (h⁻¹) |
| Exam- | 30.1 | Catalyst | Se/DMAP | 70 | 4.76 | 2 | 79.7 | 71.9 | 5.4 |
| ple 1 | | system | (1:3) | | | | | | |
| | | 1-1 | | | | | | | |
| Exam- | 100.6 | Catalyst | Se/DMAP | 70 | 4.76 | 2 | 67.4 | 65.0 | 16.3 |
| ple 2 | | system | (1:3) | | | | | | |
| | | 1-1 | | | | | | | |
| Exam- | 100.8 | Catalyst | Se | 70 | 4.76 | 2 | 22.6 | 0.3 | — |
| ple 3 | | system 2 | only | | | | | | |
| Exam- | — | Catalyst | DMAP | 70 | 4.76 | 2 | 15.1 | 0.9 | — |
| ple 4 | | system 3 | only | | | | | | |
| Exam- | 205.8 | Catalyst | Se/DMAP | 90 | 6.12 | 2 | 53.2 | 40.6 | 20.9 |
| ple 5 | | system | (1:3) | | | | | | |
| | | 1-1 | | | | | | | |
| Exam- | 200.9 | Catalyst | Se/DMAP | 90 | 6.12 | 1 | 55.8 | 41.3 | 41.4 |
| ple 6 | | system | (1:3) | | | | | | |
| | | 1-1 | | | | | | | |
| Exam- | 222.6 | Catalyst | Se/Ph₂Se₂/ | 90 | 6.12 | 1 | 65.8 | 57.3 | 63.8 |
| ple 7 | | system | DMAP | | | | | | |
| | | 4-1 | (1:1:3) | | | | | | |
| Exam- | 202.2 | Catalyst | Se/Ph₂Se₂ | 90 | 6.12 | 1 | 19.8 | 5.9 | 6.0 |
| ple 8 | | system 5 | (1:1) | | | | | | |
| Exam- | 222.1 | Catalyst | Ph₂Se₂/ | 90 | 6.12 | 1 | 24.7 | 4.0 | 4.4 |
| ple 9 | | system 6 | DMAP | | | | | | |
| | | | (1:3) | | | | | | |
| Exam- | 208.4 | Catalyst | Se/Ph₂Se₂/ | 110 | 6.12 | 1 | 41.8 | 25.7 | 26.7 |
| ple 10 | | system | DMAP | | | | | | |
| | | 4-1 | (1:1:3) | | | | | | |
| Exam- | 209.2 | Catalyst | Se/Ph₂Se₂/ | 70 | 6.12 | 1 | 54.1 | 40.5 | 42.2 |
| ple 11 | | system | DMAP | | | | | | |
| | | 4-1 | (1:1:3) | | | | | | |
| Exam- | 212.5 | Catalyst | Se/Ph₂Se₂/ | 90 | 4.76 | 1 | 36.0 | 22.3 | 23.7 |
| ple 12 | | system | DMAP | | | | | | |
| | | 4-1 | (1:1:3) | | | | | | |
| Exam- | 206.1 | Catalyst | Se/Ph₂Se₂/ | 90 | 7.48 | 1 | 41.2 | 28.7 | 29.6 |
| ple 13 | | system | DMAP | | | | | | |
| | | 4-1 | (1:1:3) | | | | | | |
| Exam- | 550.5 | Catalyst | Se/Ph₂Se₂/ | 90 | 6.12 | 1 | 53.9 | 31.8 | 87.6 |
| ple 14 | | system | DMAP | | | | | | |
| | | 4-1 | (1:1:3) | | | | | | |
| Exam- | 1045.9 | Catalyst | Se/Ph₂Se₂/ | 90 | 6.12 | 1 | 25.3 | 14.5 | 75.6 |
| ple 15 | | system | DMAP | | | | | | |
| | | 4-1 | (1:1:3) | | | | | | |
| Exam- | 1974.0 | Catalyst | Se/Ph₂Se₂/ | 90 | 6.12 | 1 | 19.2 | 5.0 | 49.3 |
| ple 16 | | system | DMAP | | | | | | |
| | | 4-1 | (1:1:3) | | | | | | |

* Reaction conditions in Table 2: MEG (75 mmol), Se (variable), DMAP (variable), and P = variable (O₂/CO = 1/4)

### Examples 17 to 22

Bis(2-methoxyethyl) carbonate was obtained in the same manner as in the above method by performing the oxidative carbonylation of 2-methoxyethanol (MEG) using various catalyst systems with different Se/DMAP molar ratios and a condition whether Ph₂Se₂ was contained. The catalyst systems used in Examples 17 to 22, Conv., yield, and turnover frequency (TOF) are specifically described in Table 3 below.

TABLE 3

| | Catalyst system | | Conversion | Yield | TOF |
|---|---|---|---|---|---|
| Entry | (molar ratio) | | (%) | (%) | (h⁻¹) |
| Example | Catalyst | Se/Ph2Se2/DMAP | 48.9 | 39.1 | 40.5 |
| 17 | system 4-2 | (1:1:1) | | | |
| Example | Catalyst | Se/Ph2Se2/DMAP | 65.8 | 57.3 | 63.8 |
| 18 | system 4-1 | (1:1:3) | | | |
| Example | Catalyst | Se/Ph2Se2/DMAP | 68.9 | 56.2 | 60.6 |
| 19 | system 4-3 | (1:1:5) | | | |
| Example | Catalyst | Se/DMAP (1:1) | 38.2 | 20.0 | 21.2 |
| 20 | system 1-2 | | | | |
| Example | Catalyst | Se/DMAP (1:3) | 55.8 | 41.3 | 41.4 |
| 21 | system 1-1 | | | | |
| Example | Catalyst | Se/DMAP (1:5) | 46.7 | 21.4 | 21.9 |
| 22 | system 1-3 | | | | |

*Reaction conditions in Table 3: MEG (75 mmol), Se (0.375 mmol), MEG/Se molar ratio = 200, Ph₂Se₂ (0.375 mmol), DMAP (variable), T = 90° C., P = 6.12 MPa (O₂/CO = 1/4), and t = 1 h

### Examples 23 to 27

Bis (2-methoxyethyl) carbonate was obtained in the same manner as in the above method by performing the oxidative carbonylation of 2-methoxyethanol (MEG) using catalyst systems to which different types of R₂Se₂ (dialkyl diselenide) serving as a promoter were applied. The catalyst systems used in Examples 23 to 27, promoters, Cony., yield, and turnover frequency (TOF) are specifically described in Table 4 below.

TABLE 4

| | Catalyst | active | | Conversion | Yield | TOF |
|---|---|---|---|---|---|---|
| Entry | system | species | Promoter | (%) | (%) | (h⁻¹) |
| Example | Catalyst | Se/DMAP | — | 55.8 | 41.3 | 41.4 |
| 23 | system | (1:3) | | | | |
| | 1-1 | | | | | |
| Example | Catalyst | Se/DMAP | Ph₂Se₂ | 65.8 | 57.3 | 63.8 |
| 24 | system | (1:3) | | | | |
| | 4-1 | | | | | |
| Example | Catalyst | Se/DMAP | Me₂Se₂ | 51.1 | 34.0 | 35.3 |
| 25 | system | (1:3) | | | | |
| | 4-4 | | | | | |
| Example | Catalyst | Se/DMAP | Bz₂Se₂ | 58.2 | 40.2 | 40.2 |
| 26 | system | (1:3) | | | | |
| | 4-5 | | | | | |
| Example | Catalyst | Se/DMAP | Ph₂Se | 51.5 | 32.5 | 32.9 |
| 27 | system | (1:3) | | | | |
| | 4-6 | | | | | |

*Reaction conditions in Table 4: MEG (75 mmol), Se (0.375 mmol), MEG/Se molar ratio = 200, DMAP (1.125 mmol), R₂Se₂ (0.375 mmol, dialkyl diselenides), T = 90° C., P = 6.12 MPa (O₂/CO = 1/4), and t = 1 h

### Examples 28 to 37

A dialkyl carbonate (DAC) was obtained in the same manner as in the above method by performing oxidative carbonylation using different types of alcohol (ROH). The types of alcohol, catalyst systems, DAC yield, turnover frequency (TOF), and selectivity to alkylated Se are specifically described in Table 5 below. The alkylated selenium is a by-product of the reaction, so the amount thereof or selectivity thereto is required to be low.

TABLE 5

| | | | | DAC ¹⁾ | | Selectivity to |
|---|---|---|---|---|---|---|
| | Alcohol | | | Yield | TOF | alkylated Se |
| Entry | (ROH) | Catalyst system | | (%)^{b} | (h⁻¹) | (%) |
| Exam- | Methanol | Catalyst | Se/Ph₂Se₂/ | 33.5 | 35.3 | 18.4 |
| ple 28 | | system | DMAP | | | |
| | | 4-1 | | | | |
| Exam- | Ethanol | Catalyst | Se/Ph₂Se₂/ | 26.1 | 25.8 | 11.1 |
| ple 29 | | system | DMAP | | | |
| | | 4-1 | | | | |
| Exam- | 1- | Catalyst | Se/Ph₂Se₂/ | 13.9 | 13.4 | 6.2 |
| ple 30 | Propanol | system | DMAP | | | |
| | | 4-1 | | | | |
| Exam- | 1- | Catalyst | Se/Ph₂Se₂/ | 11.0 | 11.0 | 3.2 |
| ple 31 | Butanol | system | DMAP | | | |
| | | 4-1 | | | | |
| Exam- | 2,2,2- | Catalyst | Se/Ph₂Se₂/ | 5.1 | 4.9 | 0.6 |
| ple 32 | Trifluoro- | system | DMAP | | | |
| | ethanol | 4-1 | | | | |
| Exam- | Phenol | Catalyst | Se/Ph₂Se₂/ | 0 | 0 | 0 |
| ple 33 | | system | DMAP | | | |
| | | 4-1 | | | | |
| Exam- | MEG | Catalyst | Se/Ph₂Se₂/ | 57.3 | 63.8 | 2.1 |
| ple 34 | | system | DMAP | | | |
| | | 4-1 | | | | |
| Exam- | MEG | Se/KHCO₃ ²⁾ | | 14.9 | 15.3 | 4.7 |
| ple 35 | | | | | | |
| Exam- | MEG | CuCl₂ | | 2.6 | 2.6 | N.A. |
| ple 36 | | | | | | |
| Exam- | MEG | Cu/Pd/N ³⁾ | | 2.1 | 2.2 | N.A. |
| ple 37 | | | | | | |

*Reaction conditions in Table 5: ROH (75 mmol), Se (0.375 mmol), ROH/Se molar ratio = 200, Ph₂Se₂ (0.375 mmol), DMAP (1.125 mmol), Se/Ph₂Se₂/DMAP molar ratio = 1:1:3, T = 90° C., P = 6.12 MPa (O₂/CO = 1/4), and t = 1 h,
¹⁾ DAC = dialkyl carbonate
²⁾ Se/K molar ratio = 1/2
³⁾ Cu/Pd/N = molar ratios of Cu(OMe)₂/PdCl₂(PPh₃)₂/NMe₄Cl and Cu/Pd/N = 5/0.1/5

### Examples 38 to 43

A dialkyl carbonate (DAC) was obtained in the same manner as in the above method by performing oxidative carbonylation with varying molar ratios of selenium and DMAP. The catalyst systems used in Examples 38 to 43, promoters, Conv., yield, and turnover frequency (TOF) are specifically described in Table 6 below.

TABLE 6

| | Catalyst system | | Conversion | Yield | TOF |
|---|---|---|---|---|---|
| Entry | (molar ratio) | | (%) | (%) | (h⁻¹) |
| Example 38 | Catalyst | Se/DMAP | 19.9 | 3.1 | 0.8 |
| | system 1-6 | (2:1) | | | |
| Example 39 | Catalyst | Se/DMAP | 51.9 | 4.9 | 1.3 |
| | system 1-2 | (1:1) | | | |
| Example 40 | Catalyst | Se/DMAP | 64.2 | 59.5 | 14.7 |
| | system 1-4 | (1:2) | | | |
| Example 41 | Catalyst | Se/DMAP | 67.4 | 65.0 | 16.3 |
| | system 1-1 | (1:3) | | | |
| Example 42 | Catalyst | Se/DMAP | 31.6 | 14.5 | 3.6 |
| | system 1-5 | (1:4) | | | |
| Example 43 | Catalyst | Se/DMAP | 69.1 | 10.0 | 2.7 |
| | system 1-3 | (1:5) | | | |

* Reaction conditions in Table 6: MEG (75 mmol), Se (0.75 mmol), DMAP (variable), T = 70° C., P = 4.83 MPa (700 psi, O₂/CO = 1/4), and t = 2 h

[Purification of Bis(2-Methoxyethyl) Carbonate]

After the oxidative carbonylation of MEG, MEG was removed from the mixture composed of BMEC, MEG, DMAP, and selenium species under reduced pressure at 90° C. using a rotary evaporator to obtain a dark orange suspension. N-hexane was added to the orange suspension to make two phases and stored in a refrigerator overnight. During storage, DMAP and dark brown selenium (a viscous liquid) settled to the bottom. After decanting the top n-hexane solution, the resulting product was evaporated to obtain a light yellow solution, seemingly containing a large amount of BMEC and a small amount of selenium species. In order to remove the selenium species from the light yellow solution, the selenium species were oxidized to SeO₂ by adding KMnO₄ to 2M HCl and then filtered to remove solid SeO₂ (white) and MnO₂ (black). Next, the remaining solution containing BMEC and aqueous HCl was evaporated at 90° C. under reduced pressure using a rotary evaporator. Lastly, a yellow liquid was obtained, and through vacuum distillation, a light yellow liquid was obtained. The light yellow liquid is pure BMEC free of selenium.

### Experimental Example

### Experimental Example 1: Effect of Base on Oxidative Carbonylation Activity of MEG

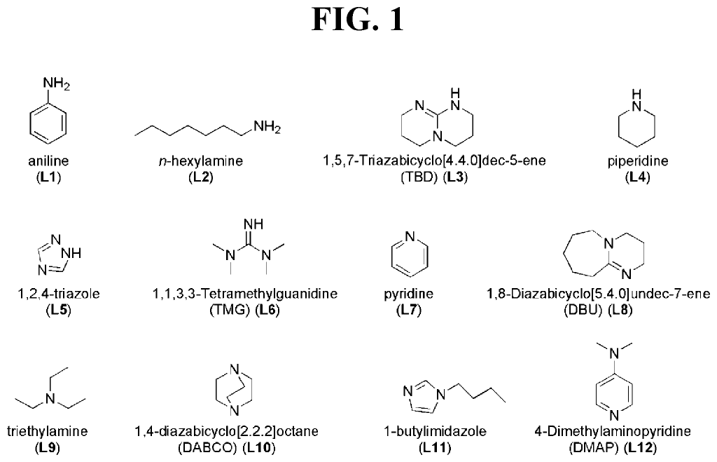

FIG. 1 is a diagram illustrating structures of base substances of L1 to L12. In the presence of bases, L8 to L12 (Se/base molar ratio=3), the reaction was performed as in Reaction Formula 2 under the condition of a MEG/Se molar ratio of 30 at 70° C. to analyze the activity of the selenium-based catalyst system for the oxidative carbonylation of 2-methoxyethanol (MEG). The results thereof are shown in Table 7 below (reaction conditions: MEG (75 mmol), Se (2.5 mmol), bases (7.5 mmol), T=70° C., P=4.76 MPa (O₂/CO=1/4), and t=2 h).

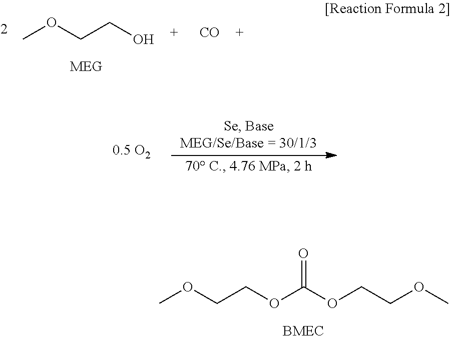

TABLE 7

| | Base | | Conversion | Yield | Selectivity | TOF |
|---|---|---|---|---|---|---|
| Entry | (pKaH) | | (%) | (%) | (%) | (h⁻¹) |
| 1 | L1 | (4.6) | 14.7 | 0 | 0.0 | 0.0 |
| 2 | L2 | (10.6) | 20.2 | 0 | 0.0 | 0.0 |
| 3 | L3 | (15.2) | 33.0 | 1.2 | 3.6 | 0.1 |
| 4 | L4 | (11.1) | 15.1 | 1.0 | 6.6 | 0.1 |
| 5 | L5 | (10.3) | 25.5 | 0.1 | 0.4 | 0.0 |
| 6 | L6 | (13.0) | 46.8 | 10.3 | 22.0 | 0.8 |
| 7 | L7 | (5.2) | 13.9 | 3.7 | 26.6 | 0.3 |
| 8 | L8 | (12.5) | 51.1 | 25.4 | 49.7 | 1.9 |
| 9 | L9 | (10.7) | 40.2 | 27.6 | 68.7 | 2.1 |
| 10 | L10 | (8.8) | 57.0 | 39.0 | 68.4 | 2.9 |
| 11 | L11 | (7.1) | 39.1 | 32.9 | 84.1 | 2.5 |
| 12 | L12 | (9.2) | 79.7 | 71.9 | 90.2 | 5.4 |
| 13 | NaOMe | (16.0) | 34.8 | 17.1 | 49.1 | 1.3 |
| 14 | KOt-Bu | (17.0) | 38.6 | 7.8 | 20.2 | 0.6 |
| 15 | KHCO₃ | (10.3) | 39.1 | 23.3 | 59.6 | 1.7 |

Referring to FIG. 1 and Table 7, while primary amines (L1 and L2) and secondary amines (L3 to L6) exhibited extremely low catalytic activities (BMEC yield of 0% to 1.20), tertiary amines (L8 to L12) exhibited relatively high yields of 25.40 to 39.0%. On the other hand, when performing the same reaction in the presence of DMAP (L12) in a base form, a MEG conversion rate of 79.70, a selectivity of 90.2%, and a BMEC yield of 71.9% were exhibited. Considering that other bases with large pKa do not exhibit better activities than DMAP, basicity cannot be said to be the most important factor affecting the activity. However, it seems that changes in basicity caused by the resonance structure of DMAP increase catalytic activity.

### Experimental Example 2: Analysis of BMEC Yield Under Various Reaction Conditions

In Table 2, the turnover frequency (TOF) is an indicator capable of determining how rapidly a catalyst with a unit mole number can produce a target material (desired material). A catalyst is considered to have good efficiency when being able to be converted into a desired material within a short period.

According to Table 2, when performing the reaction under the condition of a MEG/Se molar ratio of about 30 using the catalyst system with a Se/DMAP (1:3) molar ratio, the BMEC yield appeared to be 71.9% (TOF: 5.4 h⁻¹) (Example 1). When increasing the MEG/Se molar ratio to 100.6 under the same condition, a BMEC yield of 65.0% (TOF: 5.4 h⁻¹) was obtained (Example 2). In addition, looking into Examples 3 and 4, it was confirmed that when removing either the Se or DMAP component, the BMEC yield became extremely low, discovering that the oxidative carbonylation of MEG showed a synergistic effect when selenium (Se) and DMAP were present together. Furthermore, looking into Examples 5 and 6, the BMEC yields appeared to be 40.6% and 41.3%, respectively, and the TOF values appeared to be 20.9 h₋₁ and 41.4 h⁻¹, respectively, discovering that the TOF value was improved compared to that of Example 1. Moreover, in the case of using the Se/Ph₂Se₂/DMAP (1:1:3) catalyst system to which Ph₂Se₂ was added (Example 7), the BMEC yield appeared to be 57.3%, and the TOF value appeared to be 63.8 h⁻¹. The TOF value was not only 1.5 times higher than the result according to Example 6, but also 37.5 times higher than the result of Entry 15 (catalyst system: Se/KHCO₃) in Table 2 of Experimental Example 1. On the other hand, in the case of using the Se/Ph₂Se₂ (1:1) catalyst system and the Ph₂Se₂/DMAP (1:3) catalyst system (Examples 8 and 9), the BMEC yields appeared to be low. This means that the three components Se/Ph₂Se₂/DMAP are important factors in obtaining high BMEC yields and TOF through the oxidative carbonylation of MEG.

In addition, looking into Examples 7 and 10 to 13, when performing the reaction at different reaction temperatures and pressures using the Se/Ph₂Se₂/DMAP (1:1:3) catalyst system, it was found that the condition where the reaction temperature was 90° C. and the reaction pressure was 6.12 MPa was the most optimized conditions where the highest BMEC yield was exhibited. Furthermore, looking into Examples 7 and 14 to 16, when performing the reaction using the Se/Ph₂Se₂/DMAP (1:1:3) catalyst system by varying MEG/Se molar ratios, the maximum TOF of 87.6 h⁻¹ was able to be obtained (Example 14).

Therefore, an effective catalyst system is composed of Se and DMAP and further contains Ph₂Se₂ to promote the oxidative carbonylation of MEG, thereby obtaining an extremely high TOF.

### Experimental Example 3: Analysis of BMEC Yield According to Presence or Absence of pH₂Se₂

According to Table 3, it was found that a ratio of Se:DMAP greatly affected the activity according to the presence or absence of Ph₂Se₂. When comparing Examples 18 and 21, having the same reaction condition except for the presence or absence of Ph₂Se₂, it is found that the BMEC yield is higher in the case of Example 18 containing Ph₂Se₂. This means that the active species are Se and DMAP, and Ph₂Se₂ serves as the promoter.

### Experimental Example 4: Analysis of BMEC Yield According to Promoter

According to Table 4, Examples 25 and 26 using the catalyst systems containing dimethyl diselenide (Me₂Se₂) and dibenzyl diselenide (Bz₂Se₂), respectively, failed to exhibit activities as good as that of Example 4 using the catalyst system containing diphenyl diselenide (Ph₂Se₂), and the yields thereof appeared to be lower than that of Example 23 using the catalyst system free of the promoter. In addition, Example 27 using Ph₂Se₂ exhibited a BMEC yield of 32.5%, which was much lower than that of Example 23. This means that Ph₂Se₂ can play a role in maintaining selenium as Se(0) during the reaction or making Se metal in a nanoparticle form. In particular, while dimethyl diselenide (Ph₂Se₂) is generated as a by-product in the oxidative carbonylation of methanol, it is seen that dimethyl diselenide (Ph₂Se₂) acts as the promoter in the oxidative carbonylation of MEG in the present disclosure.

### Experimental Example 5: Oxidative Carbonylation of MEG According to Alcohol Type

According to Table 5, the reaction was performed at 90° C. with a mixed gas (CO/O₂) of 6.12 MPa for 1 hour, using Se/Ph₂Se₂/DMAP as the catalyst system, by varying types of alcohol (ROH/Se molar ratio=200). Through the carbonylation of methanol, a TOF of 35.3 h⁻¹ and a yield of dimethyl carbonate (DMC) of 33.5%, which were the highest values among the reactions in Examples 28 to 33, were obtained. However, when using methanol as a reagent, 18.4% of odorous dimethyl diselenides were produced, which was inconvenient.

In addition, the reactivity of alcohol decreased as the number of carbon atoms increased. As specific examples, Example 29 using ethanol obtained a yield of 26.1%, and Example 30 using 1-propanol and Example 31 using 1-butanol exhibited much lower yields than those of Examples 28 and 29. Furthermore, Example 32 containing 2,2,2-trifluoroethanol having an electron-withdrawing functional group exhibited extremely low reactivity and only obtained a DAC yield of 5.1%. In the case of Example 33 using phenol, no reaction occurred at all.

On the other hand, Example 34 using 2-methoxyethanol (MEG) exhibited a high BMEC yield of 57.3% under the same conditions, which means that the reactivity of alcohol was greatly affected by the electronic effect and steric effect of alcohol. On the contrary, in Example 34, alkylated Se, the by-product, was formed in an extremely small amount of 2.1%. This is a result in contrast to the case where 18.4% of the by-product is formed in Example 28 using methanol, which is determined to be attributable to the reduced alkylation ability of BMEC.

In addition, when using existing catalyst systems, such as Se/KHCO₃, CuCl₂, and Cu/Pd/N, in the carbonylation of 2-methoxyethanol (MEG) (Examples 35 to 37), the BMEC yield was analyzed to be significantly lower that of Example 34 using the Se/Ph₂Se₂/DMAP catalyst system.

### Experimental Example 6: Analysis of BMEC Yield According to Molar Ratio of Selenium and DMAP

According to Table 6, in the case of Example 41 using the catalyst system in which a molar ratio of selenium and DMAP was 1:3, the BMEC yield was the highest at 65%.

### Experimental Example 7: Evaluation of Catalyst Reuse

Table 8 below shows BMEC yields according to the number of reuse of the Se/Ph₂Se₂/DMAP (1:1:3) catalyst system. To evaluate the reusability of the catalyst according to the present disclosure, an oxidative carbonylation process using the Se/Ph₂Se₂/DMAP (1:1:3) catalyst system was performed. Then, MEG and BMEC were distilled and removed from the reaction mixture having undergone the reaction, and the solid-form selenium was filtered, washed with acetone, and dried in vacuo at room temperature. Next, MEG was injected again to test reusability.

TABLE 8

| Catalyst system | Number of use | BMEC yield (%) |
|---|---|---|
| Se/Ph₂Se₂/DMAP | Run 1 | 57.3 |
| (1:1:3) | Run 2 | 56.5 |
| (Catalyst system | Run 3 | 55.7 |
| 4-1) | Run 4 | 55.1 |
| | Run 5 | 54.2 |

According to Table 8, during BMEC synthesis, the catalyst maintained most of the initial activity thereof even when reused 5 times. This indicates that active parts of the catalyst are present in a heterogeneous state.

Although preferred embodiments of the present disclosure have been disclosed for illustrative purposes, those skilled in the art will appreciate that diverse variations and modifications are possible through addition, alteration, deletion, etc. of elements, without departing from the spirit and scope of the present disclosure. For example, each component described as a single type may be implemented to be distributed, and similarly, components described as being distributed may also be implemented in an associated form. The scope of the present disclosure is represented by the claims to be described below rather than the detailed description, and it is to be interpreted that the meaning and scope of the claims and all the changes or modified forms derived from the equivalents thereof fall within the scope of the present disclosure.

### INDUSTRIAL APPLICABILITY

A catalyst system for preparing a carbonate derivative and a method of preparing a carbonate derivative using the same system of the present disclosure enable a dialkyl carbonate to be obtained economically and feasibly at a high yield, compared to existing carbonylation processes, by performing an oxidative carbonylation process of alcohol using a selenium-based catalyst system.

In addition, the catalyst system for preparing the carbonate derivative and the method of preparing the carbonate derivative using the same system of the present disclosure have an effect of maintaining catalytic activity even when reused multiple times by using the selenium-based catalyst system.

Furthermore, while existing CuCl or CuCl₂-based catalyst systems contain halogen elements and thus cause corrosion in a reactor, the selenium-based catalyst system does not have such a problem.

1. A catalyst system for preparing a carbonate derivative, the catalyst system comprising:
selenium (Se); and
a pyridine amine compound represented by Structural Formula 1, 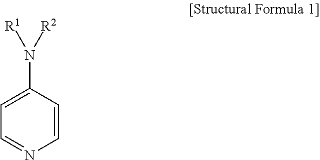
wherein in Structural Formula 1,
R¹ is an alkyl group having 1 to 3 carbon atoms, and
R² is an alkyl group having 1 to 3 carbon atoms.

2. The catalyst system of claim 1, wherein the pyridine amine compound is 4-dimethylaminopyridine (DMAP).

3. The catalyst system of claim 1, wherein in the catalyst system, a molar ratio (DMAP/Se) of 4-dimethylaminopyridine (DMAP) to selenium (Se) is in a range of 0.5 to 5.

4. The catalyst system of claim 1, further comprising a promoter.

5. The catalyst system of claim 4, wherein the promoter comprises at least one selected from the group consisting of diphenyl diselenide (Ph₂Se₂), dimethyl diselenide (Me₂Se₂), dibenzyl diselenide (Bz₂Se₂), and diphenyl selenide (Ph₂Se).

6. The catalyst system of claim 4, wherein the promoter comprises diphenyl diselenide (Ph₂Se₂).

7. The catalyst system of claim 4, wherein in the catalyst system, a molar ratio (promoter/Se) of the promoter to selenium (Se) is in a range of 0.5 to 1.5.

8. The catalyst system of claim 1, wherein the carbonate derivative is a compound represented by Structural Formula 2, 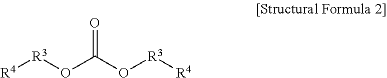
wherein in Structural Formula 2,
R³ is each independently an alkylene group having 1 to 3 carbon atoms,
R⁴ is each independently a hydrogen atom or 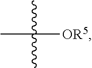
and
R⁵ is each independently an alkyl group having 1 to 3 carbon atoms.

9. The catalyst system of claim 8, wherein the carbonate derivative is bis (2-methoxyethyl) carbonate (BMEC).

10. A method of preparing a carbonate derivative, the method comprising causing reactants including Compound 3, carbon monoxide, and oxygen to react according to Reaction Formula 1 using a catalyst system to prepare Compound 2, 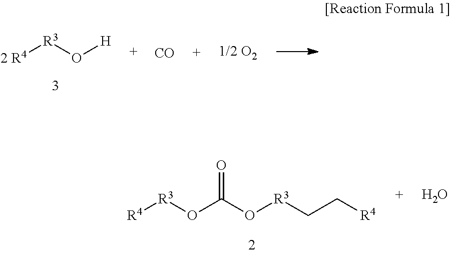
wherein in Reaction Formula 1,
R³ is each independently an alkylene group having 1 to 3 carbon atoms,
R⁴ is each independently a hydrogen atom or 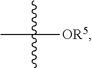
and
R⁵ is each independently an alkyl group having 1 to 3 carbon atoms.

11. The method of claim 10, wherein the catalyst system comprises selenium (Se) and a pyridine amine compound represented by Structural Formula 1, 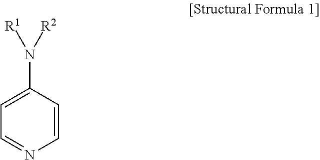
wherein in Structural Formula 1,
R¹ is an alkyl group having 1 to 3 carbon atoms, and
R² is an alkyl group having 1 to 3 carbon atoms.

12. The method of claim 11, wherein the pyridine amine compound is 4-dimethylaminopyridine (DMAP).

13. The method of claim 11, wherein the catalyst system further comprises a promoter.

14. The method of claim 13, wherein the promoter comprises at least one selected from the group consisting of diphenyl diselenide (Ph₂Se₂), dimethyl diselenide (Me₂Se₂), dibenzyl diselenide (Bz₂Se₂), and diphenyl selenide (Ph₂Se).

15. The method of claim 13, wherein the promoter comprises diphenyl diselenide (Ph₂Se₂).

16. The method of claim 10, wherein the reaction is performed at a temperature in a range of 40° C. to 150° C.

17. The method of claim 10, wherein the reaction is performed at a pressure in a range of 1 to 10 MPa.

18. The method of claim 10, wherein the reaction is performed for a duration in a range of 30 minutes to 5 hours.
